(19) United States
(12) Patent Application Publication
(10) Pub. No.: US 2024/0261545 A1
(43) Pub. Date: Aug. 8, 2024

(54) WEARABLE CANNULA TUBE HOLDER

(71) Applicant: Wanda Ruiz, Rochester, NY (US)

(72) Inventor: Wanda Ruiz, Rochester, NY (US)

(21) Appl. No.: 18/401,812

(22) Filed: Jan. 2, 2024

Related U.S. Application Data
(60) Provisional application No. 63/443,419, filed on Feb. 5, 2023.

(51) Int. Cl. A61M 25/02; A61M 60/117; A61M 60/845

(52) U.S. Cl. CPC A61M 25/02; A61M 60/117; A61M 60/845; A61M 2025/0206; A61M 2025/0213; A61M 2025/026; A61M 2210/06

(57) ABSTRACT

A wearable cannula tube holder includes a head strap having a first end and a second end and a cannula tube support member configured to allow a cannula tube to have movement in the vertical and horizontal directions when the user moves their head sideways without having the cannula tube pull away from an insertion site. The head strap includes a first attachment mechanism located at the first end, a second attachment mechanism located at the second end, and a plurality of seams to create quilted portions. The quilted portions are configured to provide air flow under the head strap when the head strap is engaged with a user. The wearable cannula tube holder may be used with a right ventricular assist device or an extracorporeal membrane oxygenation device.

### PRIORITY INFORMATION

This application claims priority from U.S. Provisional Patent Application, Ser. No. 63/443,419, filed on Feb. 5, 2023. The entire content of U.S. Provisional Patent Application, Ser. No. 63/443,419, filed on Feb. 5, 2023, is hereby incorporated by reference.

### BACKGROUND

Conventional ventricular assist devices help a heart pump blood when the pumping muscle has become weak. A right ventricular assist device (RVAD) pumps blood from the right ventricle or right atrium into the pulmonary artery and to the lungs.

The right ventricular assist device may be located outside the body of the patient, wherein a set of cannula tubes are used to connect the right ventricular assist device to the patient's heart and/or arteries.

Additionally, conventional extracorporeal membrane oxygenation (ECMO) devices help patients who need lung support. The conventional extracorporeal membrane oxygenation device uses a pump to draw venous blood, through a cannula tube, and to pump the drawn blood to an oxygenator, which removes carbon dioxide from the blood and adds oxygen to the blood. The oxygenated blood is warmed and returned to a vein or artery through another cannula tube.

FIG. 1 illustrates the connection of a right ventricular assist device to a patient. As illustrated in FIG. 1, a set of cannula tubes (30 and 35) are inserted into a patient's body 10 around the neck area. The set of cannula tubes (30 and 35) connect the patient's heart 20 and/or arteries to a right ventricular assist device (not shown).

The set of cannula tubes (30 and 35) need to be secured to prevent the cannula tubes (30 and 35) from flopping around and/or pulling away from the patient's heart 20 and/or arteries when the patient is moved.

If a conventional extracorporeal membrane oxygenation device was being hooked up to a patient, the set of cannula tubes (30 and 35) need to be secured to prevent the cannula tubes (30 and 35) from flopping around and/or pulling away from the patient's veins and/or arteries when the patient is moved.

FIG. 2 illustrates a conventional cannula tube holder 50 for either a right ventricular assist device or an extracorporeal membrane oxygenation device. As illustrated in FIG. 2, the conventional cannula tube holder 50 includes a head strap 51 that is configured to create a volume 200 for receiving a patient's head (not shown). The conventional cannula tube holder 50 further includes a cannula tube holder strap 52 that secures cannula tubes (30 and 35) to the head strap 51.

The cannula tube holder strap 52 may be a strap that uses Velcro™ for attachment to the head strap 51. In this configuration, the cannula tube holder strap 52 secures cannula tubes (30 and 35) to the head strap 51 such that the cannula tubes (30 and 35) cannot move vertically or horizontally.

The restriction of the cannula tubes (30 and 35) in the vertical and horizontal directions can cause the cannula tubes to pull away from the insertion site and/or pull away from the patient's heart, veins, and/or arteries when the patient moves their head sideways.

In other words, the conventional cannula tube holder strap 52 provides no tolerance for movement of the patient's head, thereby presenting a situation where the cannula tubes can pull away from the insertion site and/or pull away from the patient's heart, veins, and/or arteries when the patient moves their head sideways.

Conventionally, a set of cannula tubes has also been secured to the patient's body using adhesive tape or a large adhesive bandage. The adhesive tape can be uncomfortable for the patient and does not necessarily provide an adequate means for securing the cannula tubes during the movement of the patient.

Moreover, the adhesive tape or the large adhesive bandage prevents the covered area of the skin from being exposed to the air, thereby making the skin susceptible to infections and/or irritation.

Lastly, the adhesive tape or large adhesive bandage provides such a secure attachment of the cannula tubes to the patient that the cannula tubes (30 and 35) cannot move vertically or horizontally. If the patient moves their head sideways, the movement of the patient's head can cause the cannula tubes, secured by adhesive tape or large adhesive bandage, to pull away from the insertion site, and thus, pull away from the patient's heart, veins, and/or arteries such that the right ventricular assist device or the extracorporeal membrane oxygenation device is no longer properly engaged with the patient's heart, veins, and/or arteries.

It is important for a patient on a right ventricular assist device or an extracorporeal membrane oxygenation device to be able to move and/or receive physical therapy without having the cannula tubes pull away from the insertion site or from the patient's heart, veins, and/or arteries.

Thus, it is desirable to provide a wearable cannula tube holder for preventing the cannula tubes from flopping around and/or pulling away from the patient's heart, veins, and/or arteries when the patient is moved.

Moreover, it is desirable to provide a wearable cannula tube holder that is comfortable for the patient and allows for some breathability to the skin underlying the wearable cannula tube holder.

Furthermore, it is desirable to provide a wearable cannula tube holder for securing the cannula tubes, while allowing some movement of the cannula tubes, when the patient moves their head, to prevent the cannula tubes from pulling away from the insertion site or from the patient's heart, veins, and/or arteries.

Additionally, it is desirable to provide a wearable cannula tube holder for preventing the cannula tubes from flopping around and/or pulling away from the insertion site or from the patient's heart, veins, and/or arteries when the patient is moved, while allowing some movement of the cannula tubes, when the patient moves their head, and comfortable for the patient and allows for some breathability to the skin underlying the wearable cannula tube holder.

### BRIEF DESCRIPTION OF THE DRAWINGS

The drawings are only for purposes of illustrating various embodiments and are not to be construed as limiting, wherein:

FIG. 1 illustrates a conventional arrangement for cannula tubes of a right ventricular assist device or an extracorporeal membrane oxygenation device;

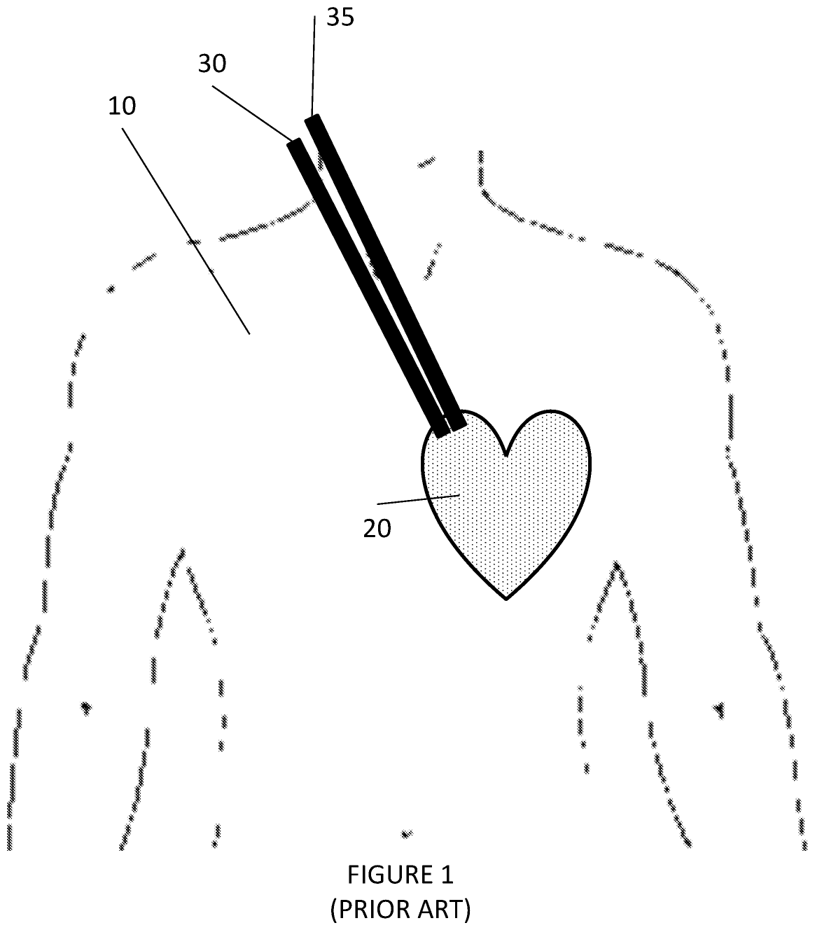

FIG. 2 illustrates a conventional cannula tube holder;

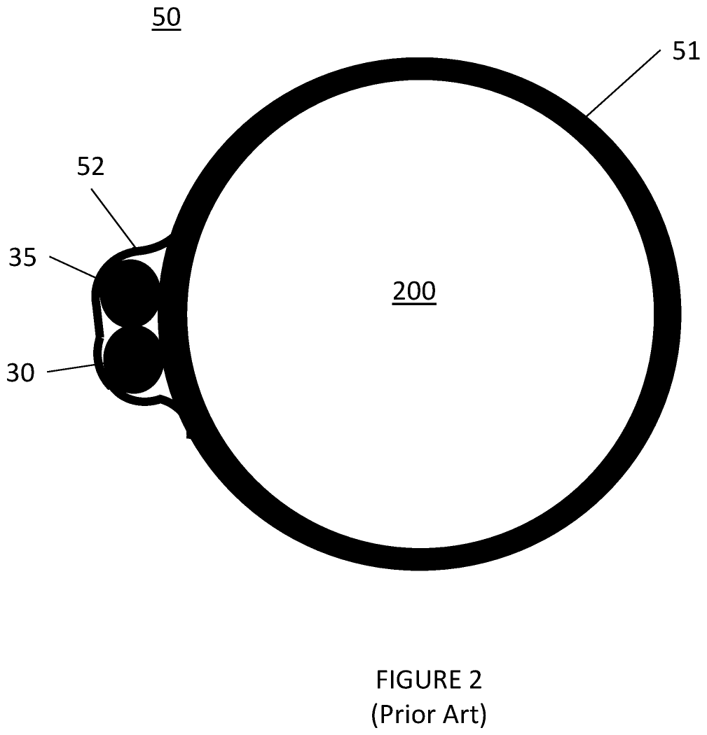

FIG. 3 illustrates a top view of an unengaged wearable cannula tube holder for securing cannula tubes to a patient's head;

FIG. 4 illustrates a bottom view of the unengaged wearable cannula tube holder of FIG. 3;

FIG. 5 illustrates a side view of the wearable cannula tube holder having a quilted configuration;

FIG. 6 illustrates a top view of the wearable cannula tube holder for securing cannula tubes to a patient's head;

FIG. 7 illustrates the wearable cannula tube holder securing cannula tubes.

### DETAILED DESCRIPTION OF THE DRAWINGS

For a general understanding, reference is made to the drawings. In the drawings, like references have been used throughout to designate identical or equivalent elements. It is also noted that the drawings may not have been drawn to scale and that certain regions may have been purposely drawn disproportionately so that the features and concepts may be properly illustrate

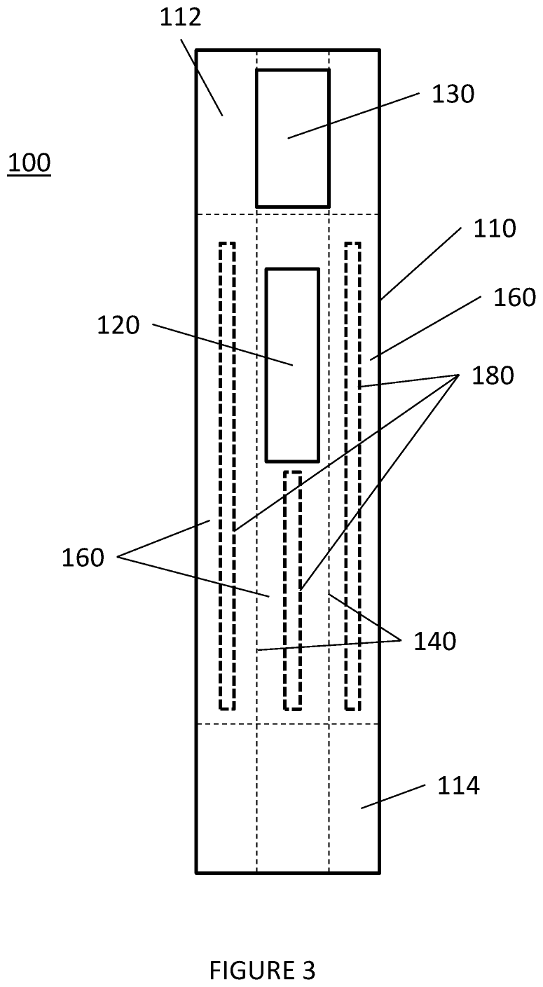

FIG. 3 illustrates a top view of an unengaged wearable cannula tube holder 100 for securing cannula tubes to a patient's head. By unengaged, a first end 112 of the wearable cannula tube holder 100 is not connected/attached to a second end 114 of the wearable cannula tube holder 100. As illustrated in FIG. 3, the first end 112 of the wearable cannula tube holder 100 is not connected/attached to a second end 114 of the wearable cannula tube holder 100.

The wearable cannula tube holder 100 includes a patient head strap 110. On the top side of the patient head strap 110, the patient head strap 110 includes a cannula tube support member 120 for securing cannula tube (not shown) to the patient head strap 110.

The cannula tube support member 120 may be a continuous open loop of material that has an opening for the cannula tube (not shown) to pass therethrough.

The cannula tube support member 120 is configured to allow the cannula tube (not shown) to have some movement in the vertical and horizontal directions such that when a patient moves their head sideways, the cannula tube (not shown) do not pull away from the insertion site or from the patient's heart, veins, and/or arteries, while still providing a secure holding mechanism to prevent the cannula tubes from flopping around when the patient is moved.

Moreover, on the top side of the patient head strap 110, the patient head strap 110, located at the first end 112 of the wearable cannula tube holder 100, includes a first attachment mechanism 130 for enabling the patient head strap 110 to be secured around a patient's head by attaching to a second attachment mechanism (not shown—located on a bottom side of the patient head strap 1) located at the second end 114 of the wearable cannula tube holder 100. The first attachment mechanism 130 may be part of a hooks and latches system, such as a Velcro™ attachment mechanism, part of a snap mechanism, or other appropriate attachment mechanism.

The patient head strap 110 may include seams 140 to create quilted portions 160. The quilted portions 160 provides additional comfort for the patient when the wearable cannula tube holder 100 engages the patient's head. Also, the quilted portions 160 provides air flow over the skin beneath the wearable cannula tube holder 100.

Lastly, as illustrated in FIG. 3, the quilted portions 160 of the patient head strap 110 includes closable openings configured to allow the user to add or remove a foam-like material (not shown) or other material that provides comfort to the patient's head when the wearable cannula tube holder 100 engages therewith.

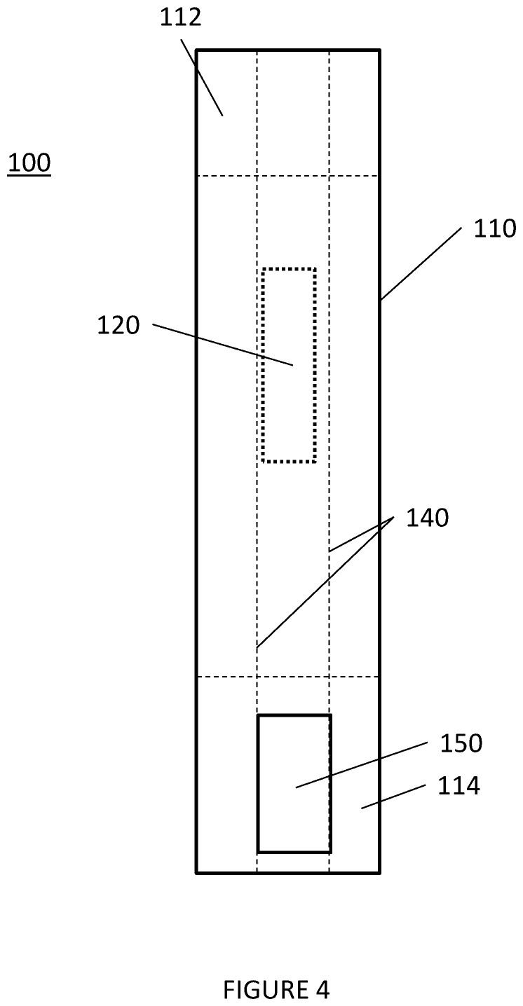

FIG. 4 illustrates a bottom view of the unengaged wearable cannula tube holder 100 of FIG. 3. By unengaged, a first end 112 of the wearable cannula tube holder 100 is not connected/attached to a second end 114 of the wearable cannula tube holder 100. As illustrated in FIG. 4, the first end 112 of the wearable cannula tube holder 100 is not connected/attached to a second end 114 of the wearable cannula tube holder 100.

On the bottom side of the patient head strap 110, the patient head strap 110, located at the second end 114 of the wearable cannula tube holder 100, includes a second attachment mechanism 150 for enabling the patient head strap 110 to be secured around a patient's head by attaching to the first attachment mechanism (not shown—located on the top side of the patient head strap 1) located at the first end 112 of the wearable cannula tube holder 100.

The second attachment mechanism 150 may be part of a hooks and latches system, such as a Velcro™ attachment mechanism, part of a snap mechanism, or other appropriate attachment mechanism.

The patient head strap 110 includes, on the top side of the patient head strap 110, a cannula tube support member 120 for securing cannula tube (not shown) to the patient head strap 110.

The cannula tube support member 120 may be a continuous open loop of material that has an opening for the cannula tube (not shown) to pass therethrough.

The cannula tube support member 120 is configured to allow the cannula tube (not shown) to have some movement in the vertical and horizontal directions such that when a patient moves their head sideways, the cannula tube (not shown) do not pull away from the insertion site or from the patient's heart, veins, and/or arteries, while still providing a secure holding mechanism to prevent the cannula tubes from flopping around when the patient is moved.

The patient head strap 110 may include optional seams 140 to create a quilted surface. The quilted surface provides additional comfort for the patient when the wearable cannula tube holder 100 engages the patient's head. Also, the quilted surface provides air flow over the skin beneath the wearable cannula tube holder 100.

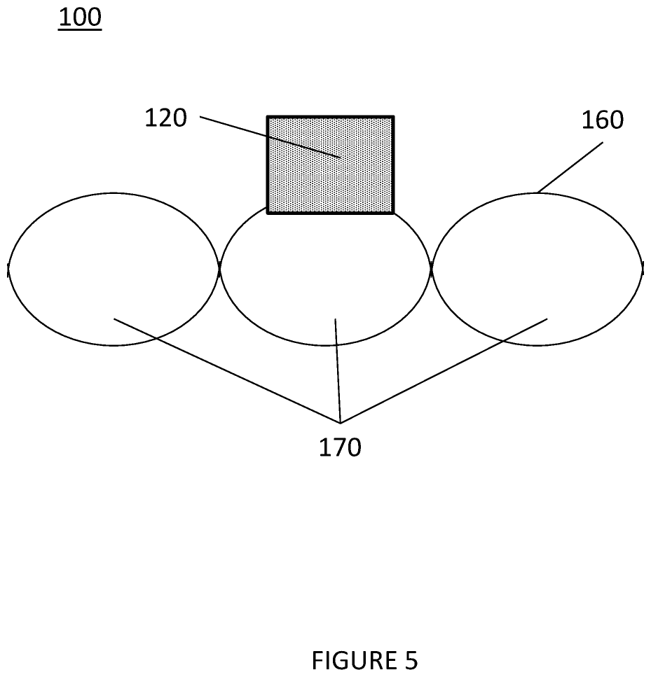

FIG. 5 illustrates a side view of a wearable cannula tube holder 100, wherein the patient head strap has a quilted configuration. As illustrated in FIG. 5, the wearable cannula tube holder 100 includes quilted portions 160, which form tubes or chambers in the patient head strap.

The quilted portions 160 can be filled with a foam-like material 170 or other material that provides comfort to the patient's head when the wearable cannula tube holder 100 engages therewith. The amount of foam-like material 170 can be varied to provide different levels of firmness or softness.

The wearable cannula tube holder 100 also includes a cannula tube support member 120 for securing cannula tube (not shown) to the patient head strap 110.

The cannula tube support member 120 may be a continuous open loop of material that has an opening for the cannula tube (not shown) to pass therethrough.

The cannula tube support member 120 is configured to allow the cannula tube (not shown) to have some movement in the vertical and horizontal directions such that when a patient moves their head sideways, the cannula tube (not shown) do not pull away from the insertion site or from the patient's heart, veins, and/or arteries, while still providing a secure holding mechanism to prevent the cannula tubes from flopping around when the patient is moved.

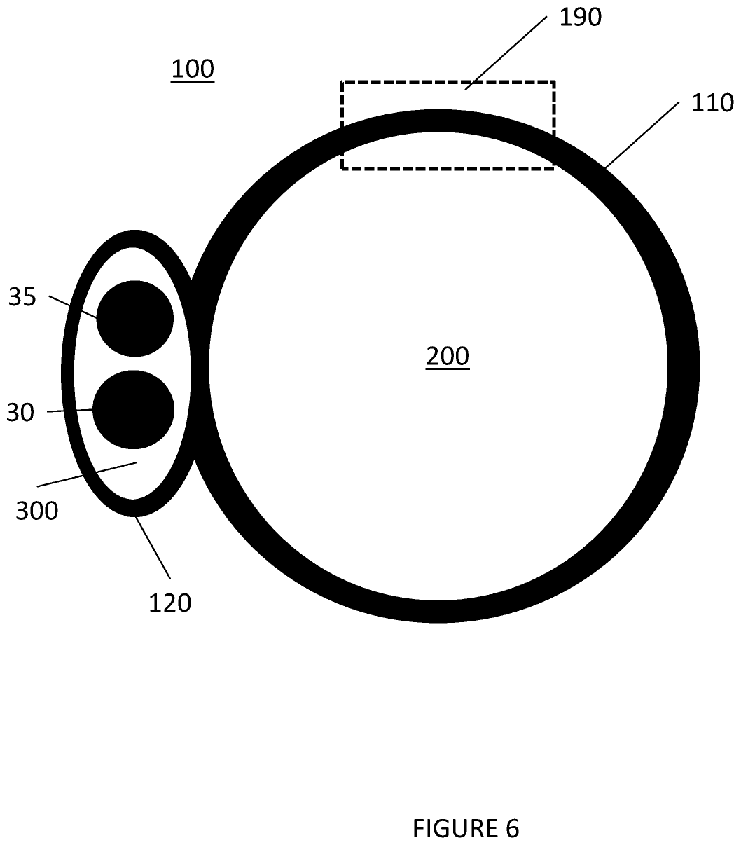

FIG. 6 illustrates a top view of a wearable cannula tube holder 100 for securing cannula tubes (30 and 35) to a patient's head, via the patient head strap 110. As illustrated in FIG. 6, a first end (not shown) of a patient head strap 110 is connected/attached to a second end (not shown) of the patient head strap 110 at an attachment location 190.

As illustrated, the attachment location 190 aligns with a back portion of a patient's head (not shown). However, the attachment location 190 can align with a front portion of the patient's head (not shown) or the left side of the patient's head (not shown).

When the first end (not shown) of the patient head strap 110 is connected/attached to the second end (not shown) of the patient head strap 110 at an attachment location 190, a volume 200 is created, wherein a patient's head (not shown) would be located therein.

Additionally, a cannula tube support member 120 for securing cannula tube (not shown) to the patient head strap 110 is preferably located on the patient head strap 110 such that the cannula tube support member 120 aligns with a right side of a patient's head (not shown). The cannula tube support member 120 forms a volume 300 for securing cannula tube (30 and 35) passing therethrough.

As illustrated, the cannula tube support member 120 may be a continuous open loop of material that has an opening for the cannula tube (not shown) to pass therethrough.

The cannula tube support member 120 is configured to allow the cannula tube (not shown) to have some movement in the vertical and horizontal directions such that when a patient moves their head sideways, the cannula tube (not shown) do not pull away from the insertion site or from the patient's heart, veins, and/or arteries, while still providing a secure holding mechanism to prevent the cannula tubes from flopping around when the patient is moved.

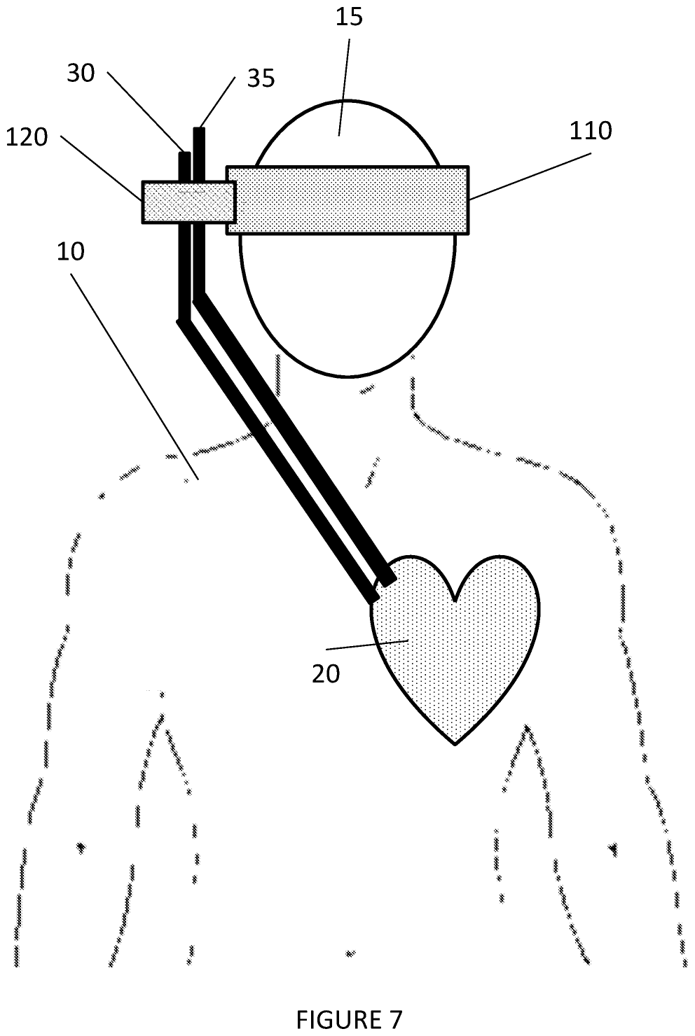

FIG. 7 illustrates the wearable cannula tube holder securing cannula tubes. As illustrated in FIG. 7, a set of cannula tubes (30 and 35) are inserted into a patient's body 10 around the neck area. The set of cannula tubes (30 and 35) connect the patient's heart 20, veins, and/or arteries to a right ventricular assist device or an extracorporeal membrane oxygenation device (not shown).

The wearable cannula tube holder includes a patient head strap 110 for encircling a patient's head 15. The patient head strap 110 includes a cannula tube support member 120 for securing cannula tubes (30 and 35) to the patient head strap 110.

The cannula tube support member 120 may be a continuous open loop of material that has an opening for the cannula tubes (30 and 35) to pass therethrough.

The cannula tube support member 120 is configured to allow the cannula tubes (30 and 35) to have some movement in the vertical and horizontal directions such that when a patient moves their head sideways, the cannula tubes (30 and 35) do not pull away from the insertion site or from the patient's heart, veins, and/or arteries, while still providing a secure holding mechanism to prevent the cannula tubes (30 and 35) from flopping around when the patient is moved.

The patient head strap 110 may include seams to create a quilted portions. The quilted portions provide additional comfort for the patient when the wearable cannula tube holder 100 engages the patient's head.

Although FIG. 7 illustrates the wearable cannula tube holder used in conjunction with a right ventricular assist device, the illustrated wearable cannula tube holder can be used in conjunction with an extracorporeal membrane oxygenation device

The patient head strap and cannula tube support member are preferably constructed of a medical grade material or fabric that is safe to use in a medical environment.

Due to the nature of the environment where the wearable cannula tube holder may be used, the wearable cannula tube holder is preferably constructed for single use as the wearable cannula tube holder may become contaminated with the bodily fluids of the patient.

The wearable cannula tube holder provides a cannula tube support member configured to allow the cannula tubes to have some movement in the vertical and horizontal directions such that when a patient moves their head sideways, the cannula tubes do not pull away pulling away from the insertion site or from the patient's heart, veins, and/or arteries, while still providing a secure holding mechanism to prevent the cannula tubes from flopping around when the patient is moved.

A wearable cannula tube holder includes a head strap having a first end and a second end and a cannula tube support member configured to allow a cannula tube to have movement in the vertical and horizontal directions when the user moves their head sideways without having the cannula tube pull away from an insertion site; the head strap including a first attachment mechanism located at the first end, a second attachment mechanism located at the second end, and a plurality of seams to create quilted portions, the quilted portions being configured to provide air flow under the head strap when the head strap is engaged with a user.

The quilted portions may include a foam-like material.

Each quilted portion may include a closable opening configured to enable adding thereto or removal therefrom of the foam-like material.

The head strap may be constructed of a medical grade material.

The head strap may be constructed of a medical grade fabric.

The cannula tube support member may be an open loop of material having an opening for the cannula tube.

The cannula tube support member may be a continuous open loop of material having an opening for the cannula tube.

The first attachment mechanism and the second attachment mechanism may be a hooks and latches attachment system.

The first attachment mechanism and the second attachment mechanism may be a snap attachment system.

It will be appreciated that variations of the above-disclosed embodiments and other features and functions, or alternatives thereof, may be desirably combined into many other different systems or applications.

Also, various presently unforeseen or unanticipated alternatives, modifications, variations or improvements therein may be subsequently made by those skilled in the art which are also intended to be encompassed by the description above and the following claims.

What is claimed is:

1. A wearable cannula tube holder comprising:
a head strap having a first end and a second end; and
said head strap including
a first attachment mechanism located at said first end,
a second attachment mechanism located at said second end, and
a plurality of seams to create quilted portions, said quilted portions being configured to provide air flow under said head strap when said head strap is engaged with a user;
a cannula tube support member configured to allow a cannula tube to have movement in the vertical and horizontal directions when the user moves their head sideways without having the cannula tube pull away from an insertion site.

2. The wearable cannula tube holder as claimed in claim 1, wherein said quilted portions include a foam-like material.

3. The wearable cannula tube holder as claimed in claim 2, wherein each quilted portion includes a closable opening configured to enable adding thereto or removal therefrom of said foam-like material.

4. The wearable cannula tube holder as claimed in claim 1, wherein said head strap is constructed of a medical grade material.

5. The wearable cannula tube holder as claimed in claim 1, wherein said head strap is constructed of a medical grade fabric.

6. The wearable cannula tube holder as claimed in claim 1, wherein said cannula tube support member is an open loop of material having an opening for the cannula tube.

7. The wearable cannula tube holder as claimed in claim 1, wherein said cannula tube support member is a continuous open loop of material having an opening for the cannula tube.

8. The wearable cannula tube holder as claimed in claim 1, wherein said first attachment mechanism and said second attachment mechanism are a hooks and latches attachment system.

9. The wearable cannula tube holder as claimed in claim 1, wherein said first attachment mechanism and said second attachment mechanism are a snap attachment system.
